(19) United States
(12) Patent Application Publication
(10) Pub. No.: US 2009/0197891 A1
(43) Pub. Date: Aug. 6, 2009

(54) Use of (4-Alkylpiperazinyl)(phenyl) methanones in the treatment of alzheimer's disease

(76) Inventors: Laurent Lecanu, McLean, VA (US); Janet Greeson, Las Vegas, NV (US); Vassilios Papadopoulos, North Potomac, MD (US)

Correspondence Address: SCHWEGMAN, LUNDBERG & WOESSNER, P.A., P.O. BOX 2938, MINNEAPOLIS, MN, 55402, US

(21) Appl. No.: 10/599,952

(22) PCT Filed: Apr. 12, 2005
(86) PCT No.: PCT/US05/12028

§ 371 Date: Oct. 3, 2007

Related U.S. Application Data
(60) Provisional application No. 60/562,643, filed on Apr. 15, 2004.

(51) Int. Cl. A61K 31/495; C12N 5/06

(52) U.S. Cl. 514/252.12; 435/375

(57) ABSTRACT

The invention provides a therapeutic method for treating at least one symptom of Alzheimer's disease in a mammal, such as a human, wherein the toxicity of a pathogen of β amyloid peptide mammalian cells is implicated and inhibition of the subsequently-induced pathological pathways is desired comprising administering to a mammal in need of such therapy, an effective amount of a benzoylpiperazine derivative, including pharmaceutically acceptable salts thereof.

### REFERENCE TO RELATED APPLICATIONS

This application claims priority under 35 U.S.C. 119(e) from U.S. Provisional Application Ser. No. 60/562,643 filed Apr. 15, 2004.

### BACKGROUND OF THE INVENTION

Alzheimer's disease (AD) is the most common dementia occurring in elderly, affecting about 10% of people above 65 years and 40% above 80 years. The familial AD is the early-onset form of the disease that involves different mutations of the amyloid protein precursor (APP) gene and accounts for no more than 5% of the total AD cases. The late-onset form of the disease, also called sporadic form, accounts for more than 95% of the AD cases and its origins remain elusive. Several risk factors have been identified or are suspected. These include the ε4 allele of the apoE gene, socio-economical situation or previous medical conditions, but a causality relationship of the onset or progression of the disease has not been yet established.

AD is clinically characterized by a progressive and irreversible impairment of cognition processes and memory alteration, and is commonly associated with a non-cognitive symptomatology, including depression (Robert et al., Alzheimer's Disease: from molecular biology to therapy, R. Becker et al., eds., (1996) at 487-493). Alzheimer's disease (AD) neuropathology is histologically characterized by an increase of brain β-amyloid (Aβ) peptide levels accompanied by the formation of senile plaques (Nikaido et al. (1970) Trans Am. Neurol. Assoc. 95:47-50) and the appearance of neurofibrillary tangles (NFT), due to a hyperphosphorylation of the Tau protein (Kosik et al., (1986) PNAS USA 83:4044-8). Aβ is produced by proteolytic cleavage of the β-amyloid precursor protein (β-APP) by the membrane enzymes β- and γ-secretase. Aβ exists either as the most commonly found 40 amino acid length Aβ₁₋₄₀ form or the 42 amino acid Aβ₁₋₄₂ form, reported to be more neurotoxic than Aβ₁₋₄₀. Although understanding of Aβ-medicated neurotoxicity has dramatically increased during the last decade, no Aβ₁₋₄₂ targeting therapeutic strategy has been shown to successfully slow down the progression of the disease. Rather, current therapeutic strategies under investigation for AD include inhibitors of Aβ production, compounds that prevent its oligomerization and fibrillization, anti-inflammatory drugs, inhibitors of cholesterol synthesis, antioxidants, neurorestorative factors and vaccines (Selkoe, D. J. (1999) Nature 399, A23-31; Emilien, G., et al. (2000) Arch. Neurol. 57, 454-459; Klein, W. L. (2002) Neurochem. Internat. 41, 345-52; Helmuth, L. (2002) Science 297(5585), 1260-21).

### SUMMARY OF THE INVENTION

The invention provides a method to treat Alzheimer's disease, for example, by blocking or inhibiting the ability of glutamate or β-amyloid, such as Aβ₁₋₄₂, Aβ₁₋₄₀ or Aβ₁₋₄₃, to damage mammalian neurons. Thus, the present invention provides a method for treatment of a mammal threatened or afflicted by Alzheimer's disease, by administering to said mammal an effective amount of a compound of formula I:

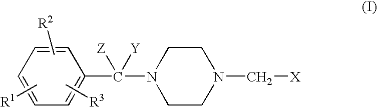

wherein:

a) R¹, R² and R³ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl, (C₁-C₆)alkylthio, thio(C₁-C₆)alkyl, (C₁-C₆)alkanoyloxy, N(R⁶)(N⁷) wherein R⁶ and R⁷ are individually H, O, (C₁-C₆) alkyl, (C₃-C₆)cycloalkyl, (C₃-C₆)cycloalkyl(C₁-C₆)alkyl, phenyl or benzyl, or R⁶ and R⁷, together with the N to which they are attached, form a 5- or 6-membered ring which may optionally contain 1-2 S, N(R⁶) or nonperoxide O; or R¹ and R² together are methylenedioxy;

b) Y and Z together are ═O, —O(CH₂)ₘO— or —CH₂)ₘ— wherein m is 2-4, or Y is H and Z is OR⁹ or SR⁹, wherein R⁹ is H or (C₁-C₄)alkyl;

c) X is (C₁-C₆) alkyl, (C₁-C₆)alkoxy, hydroxy(C₁-C₆)alkyl (C₃-C₁₂) alkenyl, (C₂-C₆) alkynyl, carboxy, (C₁-C₆)alkoxycarbonyl, thio(C₁-C₆) alkyl, (C₁-C₆)alkylthio, (C₃-C₁₂)heterocyclo, (C₃-C₁₂) heterocycloalkyl (C₁-C₆) alkyl, aryl or heteroaryl, optionally substituted by 1, 2 or 3 R¹;

and the pharmaceutically acceptable salts thereof.

Preferably, at least one of R¹, R² or R³ is not H, e.g., 1, 2 or 3 of R¹, R² and R³ are not H.

Preferably R¹ is (C₁-C₆) alkoxy; e.g., (C₁-C₃) alkoxy, preferably in the 4-position.

Preferably R¹ and R² are (C₁-C₆) alkoxy, e.g. (C₁-C₃) alkoxy, preferably in the 3,4-positions.

Preferably R¹, R² and R³ are (C₁-C₆) alkoxy, e.g., (C₁-C₃) alkoxy, preferably in the 2,3, and/or 4-positions or two of R¹, R² and R³ are methylenedioxy.

Preferably Z and Y together are ═O (oxo).

Preferably X is (C₁-C₆)alkyl; e.g., (C₁-C₃)alkyl, such as CH₃ or CH₂CH₃; or X is CH[(C₁-C₆)alkyl] [CO₂Q] wherein Q is H or (C₁-C₆)alkyl.

Preferably X is (C₃-C₁₂)heterocyclo.

The invention also provides a pharmaceutical composition such as a unit dosage form, comprising a compound of formula I, or a pharmaceutically acceptable salt thereof, in combination with a pharmaceutically acceptable diluent or carrier, which optionally can include one or more anti-AD agents of one or more of the classes of anti-AD agents referenced hereinabove, and can optionally include stabilizers, preservatives, and absorption control agents.

Additionally, the invention provides a therapeutic method for preventing or treating a pathological condition or symptom in a mammal, such as a human, that is associated with AD or the onset of AD, or that is associated with the toxicity of a pathogen such as β-amyloid peptide and/or glutamate toward mammalian neuronal cells, wherein inhibition of said toxicity is desired, or down-modulation of the subsequently induced pathological pathway is desired, comprising administering to a mammal in need of such therapy, an effective amount of a compound of formula I, or a pharmaceutically acceptable salt thereof.

Thus, the invention also provides a therapeutic method to treat a neuropathy that involves glutamate network hyperactivity, such as cerebral ischemia, AIDS-associated dementia, stroke, traumatic brain or spinal chord injury, and the like.

The invention provides a compound of formula I for use in medical therapy (e.g., for use in treating a mammal afflicted or threatened with AD, as well as the use of a compound of formula I for the manufacture of a medicament useful for the treatment of at least one AD symptom in a mammal, such as a human, such as an AD patient.

The invention also provides novel compounds of formula I, as well as, processes and intermediates disclosed herein that are useful for preparing compounds of formula (I) or salts thereof. This includes analogs in which the C(Y)(Z) group is bound to a carbon atom of R¹, R² or R³ or to a CH₂ group of piperazine or homopiperazine. Many of the compounds of formula I are also useful as intermediates in the preparation of compounds of formula I.

### SUMMARY OF THE FIGURES

FIG. 1 depicts the chemical formula of procaine and of certain procaine derivatives. SP015, SP016 and SP017 were identified by screening a natural compounds database using procaine and procainamide as a substructure.

FIG. 2 (panels A-C) are graphs depicting the effect of Aβ₁₋₄₂ on rat pheochromocytoma PC12 cells; cell viability was assessed by MTT assay (A) and by measuring the intracellular ATP concentrations (B). The effect of Aβ₁₋₄₂ on the free radical production was assayed using the fluorescent probe 2,7-DCF (C). PC12 cells were exposed to increasing concentrations of Aβ₁₋₄₂ (C=control) and the different parameters were assayed after 24 hours exposure. The statistical analysis was performed using one-way ANOVA followed by Dunnett's test. Mean±SD, n=6. * p<0.05, *** p<0.001 compared to control unless differently specified.

FIG. 3 (panels A-F) are graphs depicting the effect of procaine and SP008 on cell viability and Aβ₁₋₄₂ induced ATP depletion in PC12 cells. PC12 cells were pre-incubated with increasing concentrations of procaine or SP008 for 24 hours before being exposed to increasing concentrations of Aβ₁₋₄₂ for 24 hours. Cell viability was assessed by MTT assay (A, B, C) and the free radical production was measured using the fluorescent probe 2,7-DCF (D, E, F). The cell viability results are presented as inhibition percentage of the NADPH-diaphorase activity, considering that the 100% inhibition corresponds to the effect observed with Aβ₁₋₄₂: The statistical analysis was performed using one-way ANOV-A followed by Dunnett's test. Mean±SD, n=6. * p<0.05, ** p<0.001 compared to vehicle group unless differently specified.

FIG. 4 is a graph depicting the neuroprotective effect of procaine and SP008 against glutamate-induced cell death of PC12 cells. PC12 cells were pre-incubated with increasing concentrations of procaine or SP008 and 24 hours before being exposed to 100 μM glutamate for 24 hours. Cell viability was assessed by MTT assay. The statistical analysis was performed using one-way ANOVA followed by Dunnett's test. Mean±SD, n=6. ** p<0.01 compared to 0 μM. ^{xxx} p<0.001 compared to control group.

### DETAILED DESCRIPTION OF THE INVENTION

Local anesthetics have been shown to exhibit neuroprotective properties in vivo, during cerebral ischemia in gerbils (Fujitani et al. (1994), Neurosci. Lett., 179:91-4; Chen et al. (1998) Brain Res., 4:16; Adachi et al. (1999) Brit. J. Anaesth; 83:472), and in vitro, during an hypoxic episode in hippocampal neurons (Lucas et al. (1989) J. Neurosci. Methods, 28:47; Liu et al. (1997) Anesthesiology, 87:1470; Raley-Susman et al., (2001) J. Neurophysiol. 86:2715-26). Concomitantly, procaine and lidocaine have been show to inhibit NMDA receptor activity (Nishizawa et al., (2002) Anesth. Analg., 94:325-30), suppress the anoxia-induced increase of the intracellular calcium concentration in gerbil hippocampus (Liu et al., (1997) Anesthesiology, 87:1470) and prevent the ischemia-triggered increase of extracellular concentration in gerbil brain (Fujitani et al., 1994, cited above).

Although the high metabolism rate of procaine to p-aminobenzoic acid and diethylaminoethanol, by various esterases present in the blood, may explain the short duration of the presence of procaine in the body as well as its local anesthetic effect, it provides a challenge for the use of this molecule in the therapy of chronic diseases. This consideration led to the screening of a database of natural compounds using procaine as the lead structure, to identify stable biologically active analogs and discern the common chemical structure bearing the activity. The present invention thus is directed to characterization, design, synthesis, and pharmacological activity of (4-alkyl-piperazin-1-yl)-phenylmethanone derivatives which exhibit neuroprotective properties when contacted with mammalian cells. More specifically, the present invention provides (4-alkyl-piperazin-1-yl)-phenylmethanone derivatives with neuroprotective properties against β-amyloid-induced toxicity.

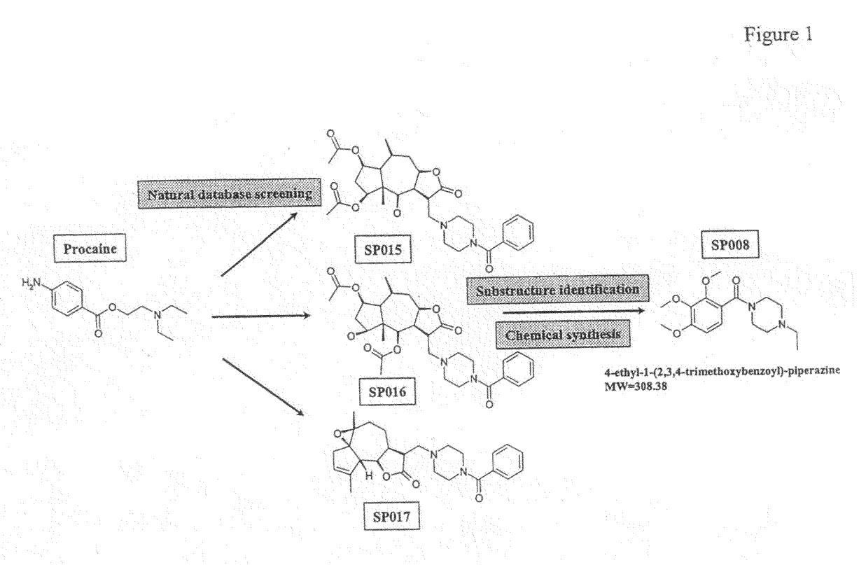

As shown in FIG. 1, 4-ethylpiperazin-1-yl-(2,3,4-trimethoxylphenyl)-methanone (SP008) is a common sub-structure derived from the local anesthetic procaine. This sub-structure is shared by molecules (SP015, SP016, SP017) isolated from plants from the Asteraceae genus, that are traditionally used to restore lost or declining mental functions. As do procaine and the SP natural compounds, SP008 displays strong neuroprotective properties against the amyloid peptide Aβ₁₋₄₂ and preserved Aβ₁₋₄₂-induced ATP depletion on rat pheochromocytoma PC12 cells, suggesting a mitochondrial site of action. Procaine and SP008 also inhibited the neurotoxic effect that glutamate displays on CP12 cells. That effect might account for the “anti-amyloid” effect observed, as the Aβ₁₋₄₂ peptide has been described to induce damaging hyper-activity of the glutamate network in neuronal cells. In addition, procaine was found to be a sigma-1 receptor ligand (IC50=4.3 μM). That receptor has been shown to protect mitochondrial functions and to have anti-depressant effects. The chemical homology suggests such a pharmacological profile for SP008. For these reasons, it is believed that SP008 and analogs thereof and of formula I can be used to treat AD.

As used herein, the term “treatment of Alzheimer's disease” includes inhibiting the development of AD in a subject exhibiting at least one of the symptoms of the onset of AD, or who is likely to develop AD, as well as the ability to halt or slow the progression of AD, or to reduce or alleviate at least one of the symptoms of AD. The term “treatment” as used with respect to any neuropathology is also intended to be defined in this manner.

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing about 5 or 6 ring atoms consisting of carbon and one to four heteroatoms each selected from the group consisting of non-peroxide oxygen, sulfur, and N(R⁶) wherein R⁶ is absent or is as defined above; as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₁₂)cycloalkyl can be monocyclic, bicyclic or tricyclic and includes cyclopropyl, cyclobutyl, cyclopentyl, cyclohexyl, bicyclo[2.2.2]octanyl, norbornyl adamantyl as well as various terpene and terpenoid structures. (C₃-C₁₂)cycloalkyl(C₁-C₆)alkyl includes the foregoing cycloalkyl and can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl. Heterocycloalkyl and (heterocycloalkyl)alkyl include the foregoing cycloalkyl wherein the cycloalkyl ring system is monocyclic, bicyclic or tricyclic and optionally comprises 1-2 S, non-peroxide O or N(R⁶) as well as 2-12 ring carbon atoms; such as morpholinyl, piperidinyl, piperazinyl, indanyl, 1,3-dithian-2-yl, and the like; The cycloalkyl ring system optionally includes 1-3 double bonds or epoxy moieties and optionally is substituted with 1-3 OH, (C₁-C₆)alkanoyloxy, (CO), (C₁-C₆)alkyl or (C₂-C₆)alkynyl. (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be formyl, acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be alkyl substituted with 1 or 2 OH groups, such as alkyl substituted with 1 or 2 OH groups, such as hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 3,4-dihydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, indanyl, or naphthyl; and heteroaryl can be furyl, imidazolyl, triazolyl, triazinyl, oxazolyl, isoxazolyl, thiazolyl, isothiazolyl, pyrazolyl, pyrrolyl, pyrazinyl, tetrazolyl, pyridyl, (or its N-oxide), thienyl, pyrimidinyl (or its N-oxide), 1H-indolyl, isoquinolyl (or its N-oxide) or quinolyl (or its N-oxide).

Compounds of formula I can be prepared as shown in Scheme A, below.

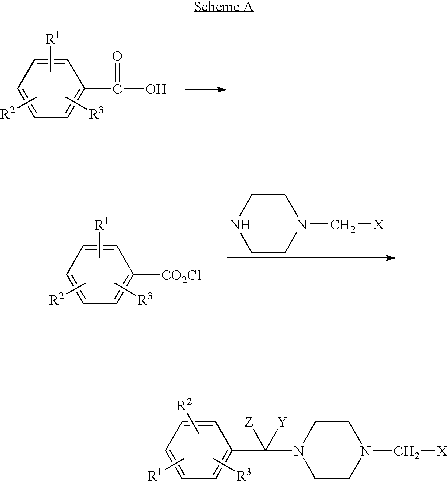

Groups R¹, R² and/or R³ on phenyl that are reactive with SOCl₂, or (C(O)Cl)₂ such as hydroxy-containing or thio-containing groups can be protected with removable protecting groups such as ethyoxyethyl, THP, (C₁-C₄)₃silyl and the like. Protected OH and hydroxylalkyl groups can be deprotected, and converted into halo, CN, alkoxycarbonyl, alkanoyloxy and alkanoyl by methods known to the art of organic synthesis. Protected amino groups can be deprotected and converted into N(R⁶)(R⁷) by methods known to the art. If necessary the C═O group can be protected and/or reduced during these conversions, then deprotected and reoxidized to C═O. See, for example, I. T. Harrison, Compendium of Organic Synthetic Reactions, Wiley-Interscience, N.Y. (1971); L. F. Fieser et al., Reagents for Organic Synthesis, John Wiley & Sons, Inc., N.Y. (1967), and U.S. Pat. No. 5,411,965.

Thus, a specific value for R¹, R², or R³ in formula I, above is H, (C₂-C₄)alkyl, N(R⁶)(R⁷), (C₂-C₄)alkoxy or (C₃-C₆)heterocycloalkyl.

A specific value for N(R⁶)(R⁷) is amino, diethylamino, dipropylamino, cyclohexylamino, or propylamino, thus a specific value for R³ is NH₂.

A preferred compound of the invention is SP008 (FIG. 1).

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium), alkaline earth metal (for example calcium or magnesium) or zinc salts can also be made.

The compounds of formula I can be formulated as pharmaceutical compositions and administered to a mammal, such as a human patient in a variety of forms adapted to the chosen route of administration, i.e., orally or parenterally, by intravenous, intramuscular, topical or subcutaneous routes, or by inhalation or insulation.

Thus, the present compounds may be systemically administered, e.g., orally, in combination with a pharmaceutically acceptable vehicle such as an inert diluent or an assimilable edible carrier. They may be enclosed in hard or soft shell gelatin capsules as powders, pellets or suspensions or may be compressed into tablets. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices, such as patches, infusion pumps or implantable depots.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection, infusion or inhalation can include sterile aqueous solutions or dispersions. Sterile powders can be prepared comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate, cellulose ethers, and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 mg to as much as 1-3 g, conveniently 10 to 1000 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline. For example, as much as about 0.5-3 g of a compound of formula I can be dissolved in about 125-500 ml of an intravenous solution comprising, e.g., 0.9% NaCl, and about 5-10% glucose. Such solutions can be infused over an extended period of up to several hours, optionally in conjunction with other anti-viral agents, antibiotics, etc. The active ingredient can also be orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as an antiviral agent may be determined using pharmacological models which are well known to the art, or using tests described below.

The following illustrate representative pharmaceutical dosage forms, containing a compound of formula I, for therapeutic or prophylactic use in humans.

| (i) Tablet 1 | mg/tablet |
|---|---|
| SP008 | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| SP008 | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| SP008 | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/ml) | mg/ml |
| SP008 (free base form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |
| (v) Iniection 2 (10 mg/ml) | mg/ml |
| SP008 (free base form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |
| (vi) Aerosol | mg/can |
| SP008 | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The invention will be further described by reference to the following detailed examples, wherein Aβ₁₋₄₂ peptide was purchased from American Peptide Co. (Sunnyvale, Calif.). Procaine, tetracaine, lidocaine, procainamide, the antioxidant tert-butyl-phenylnitrone (PBN), the N-methyl-D-aspartate (NMDA) receptor antagonist (+)-MK801, ryanodine and tetrodotoxine (TTX) were purchased from Sigma (St. Louis, Mo.). Structures of procaine, tetracaine, lidocaine, procainamide SP015, SP016 and SP017 are shown in FIG. 1. SP008 was synthesized by Taros, Inc. (Marburg, Germany) as described below. Cell culture supplies were purchased from GIBCO (Grand Island, N.Y.) and cell culture plasticware was from Corning (Corning, N.Y.) and Packard BioSciences Co. (Meriden, Conn.). RNA STAT-60 was from TEL-TEST, Inc. (Friendswood, Tex.). TaqMan® Reverse Transcription Reagents, random hexamers, and SYBR® Green PCR Master Mix were from Applied Biosystems (Foster City, Calif.).

### Methodology

### A. In Silico Screening for Procaine Derivatives

The Interbioscreen Database of naturally occurring entities was screened for compounds containing the procaine structure using the ISIS software (Information Systems, Inc., San Leandro, Calif.). Acetic acid 7-acetoxy-3-(4-benzoyl-piperazin-1-yl-methyl)-5-hydroxy-4a,8-dimethyl-2-oxo-dodecahydro-azuleno[6,5-b]furan-4-yl ester (SP015), acetic acid 5-acetoxy-3-(4-benzoylpiperazin-1-yl-methyl)-4-hydroxy-4a,8-dimethyl-2-oxo-dodecahydro-azuleno[6,5-b]furan-7-yl ester (SP016) and 3-(4-benzoyl-piperazin-1-yl-methyl)-6,6a-epoxy-6,9-dimethyl-3a,4,5,6,6a,7,9a,9b-octahydro-3H-azuleno[4,5-b]furan-2-one (SP017) compounds identified were purchased from Interbioscreen (Moscow, Russia) (FIG. 1).

### B. Cell Culture and Treatments

PC12 cells (rat pheochromocytoma) (ATCC, Manassas, Va.) were cultured in RPMI 1640 without glutamine medium containing 10% of bovine serum and 5% of horse serum at 37° and 5% CO₂. These cells respond reversibly to NGF by induction of the neuronal phenotype. PC12 cells were incubated for 24 hours in 96-well plates (5.10⁴ cells per well) with increasing concentrations (1, 10 and 100 μM) of procaine, procainamide, lidocaine, tetracaine, SP015, SP016, SP017 or SP008. Aβ₁₋₄₂ was incubated overnight at 4° C. and then added to the cells at 0.1, 1 or 10 μM final concentrations for a 24 hours time period.

To study the role played by the NMDA receptor in the Aβ₁₋₄₂-induced neurotoxicity, increasing concentrations of (+)-MK801 were added to the cell media immediately before Aβ₁₋₄₂. Cell viability was assessed 4 hours later using the MTT assay. To assess the effect of procaine and SP008 on the glutamate-induced excitotoxicity, PC12 cells were pre-treated with procaine or SP008 at 0.3, 1, 3, 10 and 30 μM for 24 hours and then submitted to glutamate exposure for another 24 hour time period. Cell viability was subsequently assessed using the MTT assay. To assess the role of sodium channels in A8142-induced neurotoxicity, PC12 cells were incubated for 4 hours with the sodium-channel blocker TTX at 3, 30 or 300 μM followed by addition of Aβ₁₋₄₂. Cell viability was assessed by MTT 24 hours later. The involvement of the oxidative stress in the toxicity of Aβ₁₋₄₂ was assessed by incubating the PC12 in the presence of 10, 100 or 500 μM PBN for 24 hours. Aβ₁₋₄₂ was then added to the incubation media. Cell viability was assessed by MTT 24 hours later.

### C. Cell Viability Determination

The cellular toxicity of Aβ was assessed using the 3-(4,5-dimethylthiazol-2-yl)-2,5-diphenyl tetrazolium bromide (MTT) assay (Trevigen, Gaithersburg, Md.) as previously described (Lecanu et al. (2004) Steroids, 69: 1-16). Briefly, 10 μl of the MTT solution were added to the cells cultured in 100 μl of medium. After an incubation period of 4 hours in the same conditions as above, 100 μl of detergent were added and cells incubated overnight at 37° C. The blue coloration was quantified at 600 nm and 690 nm using the Victor spectrophotometer (EGG-Wallac, Gaithersburg, Md.). The effect of Aβ₁₋₄₂ was expressed as (DO₆₀₀-DO₆₉₀). To compare the protective effect of the compounds tested, the decrease of MTT signal observed with Aβ₁₋₄₂ was considered to be the 100% inhibition of the NADPH diaphorase activity and the effect of the compounds tested is shown as an increase or decrease of this percentage.

### D. ATP Measurement

ATP concentrations were measured using the ATPLite-M™ assay (Packard BioSciences Co.), as previously described (Lecanu et al., cited above). In brief, cells were cultured on black 96-well ViewPlate™ and the ATP concentrations measured on a TopCount NXT™ counter (Packard BioSciences Co.) according to the manufacturer recommendations. The effect of Aβ₁₋₄₂ was expressed in arbitrary units. To compare the potential protective effect of the compounds tested on ATP recovery, the decrease of ATP concentration induced by Aβ₁₋₄₂ was considered to be 100% reduction and the effects of the compounds tested are shown as changes of this percentage.

### E. Free Radical Production

Oxidative stress was assessed by measuring the free radical production using the fluorescent probe di-hydroxy di-chlorofluorescein diacetate (2,7-DCF) (Molecular Probes, Eugene, Oreg.), as previously described (Lecanu et al., cited above). For these experiments, cells were cultured in polylysine coated microplates. Cells were washed once with RPMI 1640 and medium was then replaced by 100 μl RPMI 1640. Cells were incubated 45 minutes at room temperature in the dark with 100 μl of 2,7-DCF 50 μM and the fluorescence (excitation λ=485 nm, emission λ=535 nm) was measured using the Victor multilabel counter (EGG-Wallac, Gaithersburg, Md.).

### F. Radioligand Binding Studies

Radioligand binding studies were performed using human recombinant sigma-1 receptor expressed in Jurkat cells. Increasing concentrations of procaine ranging from 3.0E-10 to 1.0E-05 M were incubated for 120 minutes at 22° C. in presence of the specific sigma-1 receptor ligand [³H]-(+)-pentazocine at 8 nM to determine procaine IC50 and Hill value nH.

### G. Real-Time Quantitative RT-PCR (Q-PCR)

PC12 cells cultured in 6-well plates for 18 hours were treated with increasing concentrations of procaine for the indicated time period. After treatment, cells were exposed to of Aβ₁₋₄₂ 1 μM for 24 hours. At the end of the incubation, total cell RNA was extracted using RNASTAT-60 (Tel-Test, Inc., Friendswood, Tex.) according to the manufacturer's instructions. HMG-CoA reductase mRNA was quantified by Q-PCR using the ABI Prism 7700 sequence detection system (Perkin-Elmer/Applied Biosystems, Foster, City, Calif.). RT reaction was performed using TaqMan® Reverse Transcription Reagents with 1 μg total RNA and random hexamers as primers for each reaction, as previously described (Xu et al. (2003) J. Pharmacol. Ther., 307:1148-57). For quantifying rat HMG-CoA reductase mRNA with Q-PCR, the primers were designed according to GenBank Accession Number BC 019782 using PE/AB Primer Express software, which is specifically designed for the selection of primers and probes. The forward primer was 5′-GAC TGT GGT TTG TGA AGC TGT CAT-3′ (24 nucleotides; SEQ ID NO:1) and reverse primer was 5′-AAT ACT TCT CTC ACC ACC TTG GCT-3′ (24 nucleotides; SEQ ID NO:2), respectively. The primers were synthesized by BioSynthesis, Inc. (Lewisville, Tex.). Reactions were performed in a reaction mixture consisting of a 20 μl solution containing 10 μl SYBR® Green PCR Master Mix and 1 μl primers mix (5 μM each) with 2 μl cDNA. The cycling conditions were: 15 seconds at 95° C. and 1 minute at 60° C. for 40 cycles following an initial step of 2 minutes at 50° C. and 10 minutes at 95° C. AmpliTaq Gold polymerase was activated at 95° C. for 10 minutes. The 18S RNA was amplified at the same time and used as an internal control. To exclude the contamination of unspecific PCR products such as primer dimmers, a melting curve analysis was applied to all final PCR products after the cycling protocol. Also, PCR reactions without the RT reaction were performed for each sample in order to exclude genomic DNA contamination. The PCR products were collected and run on a 3% (w/v) agarose/TAE gel to confirm the product size. The threshold cycle (Ct) values for 18S RNA and samples were calculated using the PE/AB computer software. Ct was determined at the most exponential phase of the reaction. Relative transcript levels were calculated as x=2M′, in which ΔΔCt=ΔE−ΔC, and ΔE=Ctₑₓₚₑᵣᵢₘₑₙₜ−Ct_{18S}, ΔC−Ct_{control}−Ct_{18S}.

### H. Statistical Analysis

Data are expressed as mean±SD. Data obtained were assessed between experimental groups by a one-way ANOVA and Dunnett's test was used for comparison. A difference was considered significant when p<0.05.

### Example 1

### SP008 Synthesis

### 1. Materials and Methods

Solvents were purified by standard methods. MS: Recorded on a VG Tribid, Varian CH7 (EI). Thin-layer chromatography (TLC) analyses were performed on silica gel 60 F₂₅₄ with a 0.2 mm layer thickness. NMR-spectroscopy: Bruker AMX300. All resonances are given in ppm and referenced to residual solvent signals (CDCl₃: 7.25 ppm).

### 2. 2,3,4-Trimethoxybenzoyl Chloride

2,3,4-Trimethoxybenzoic acid (5.00 g, 23.6 mmol) was dissolved in dry toluene (2 mL). A catalytic amount of N,N-dimethylformamide (2 drops) was added. To this mixture was added dropwise a solution of oxalyl chloride (4.27 g, 33.6 mmol) in toluene (11 mL). Stirring was continued at room temperature for 3.5 hours. Excess reagent and solvents were removed in vacuum (yield: 5.13 g product, 94%).

¹H NMR (CDCl₃) δ 7.82 (D, 1 h, 9 Hz), 6.68 (d, 1H, 9 Hz), 3.89 (s, 3H), 3.80 (s, 1H), MS (EI) m/z 230 (M⁺), 212, 195, 179, 152.

3. 4-Ethyl-1-(2,3,4-trimethoxybenzoyl)-piperazine SP008

To a solution of crude 2,3,4-trimethoxybenzoyl chloride (0.93 g, 4.0 mmol) in dry dichloromethane (40 mL) was added drop wise N-ethylpiperazine (0.92 g, 8.1 mmol) at 0° C. Stirring was continued for 30 minutes. The mixture was washed with saturated aqueous NH₄Cl. The aqueous layer was extracted twice with dichloromethane. The combined organic layers were washed with brine, dried (MgSO₄) and concentrated. The crude product was recrystallized from ether/petroleum ether to give SP008 as a solid (0.63 g, 51%).

¹H NMR (CDCl₃) δ 6.88 (D, 1H, 8.5 Hz), 6.62 (d, 1H, 8.5 Hz), 3.83 (s, 3H), 3.81 (s, 3H), 3.80 (s, 3H), 3.76 (m, 2H), 3.25 (m, 2H), 2.43 (m, 4H), 2.35 (q, 2H, 7 Hz), 1.02 (t, 3H, 7 Hz); MS (EI) m/z 308 (M⁺), 237, 195, 97.

### Example 2

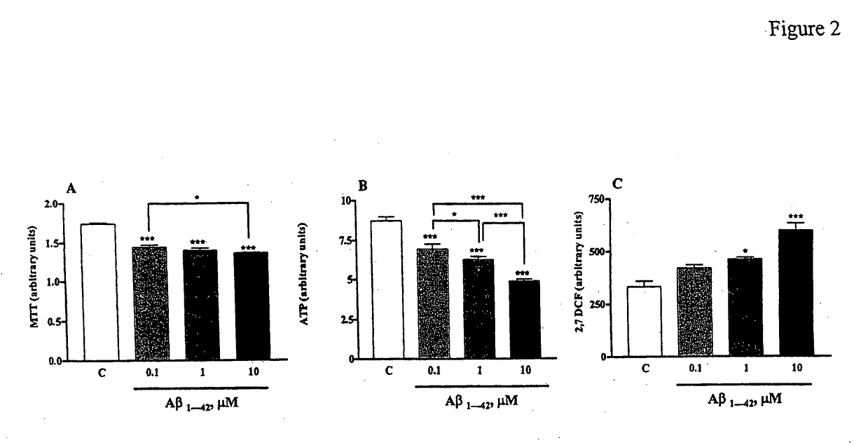

### Aβ₁₋₄₂ Neurotoxicity Assessed by MTT Assay, ATP Measurement and Free Radical Production in PC12 Cells (FIG. 2)

Aβ₁₋₄₂ induces a dose-dependent decrease of PC12 cell viability (p<0.001) (FIG. 2A) and of the intra-cellular ATP concentrations (p<0.001) (FIG. 2B). A dose-dependent relationship is also observed on the free radical production as Aβ₁₋₄₂ at 1 and 10 μM concentrations induced a significant increase of the oxidative stress (p<0.01 and p<0.001 respectively) (FIG. 2C).

### Example 3

### Effect of SP008 on Cell Viability and ATP Level of PC12 Cells Exposed to Increasing Concentrations of Aβ₁₋₄₂

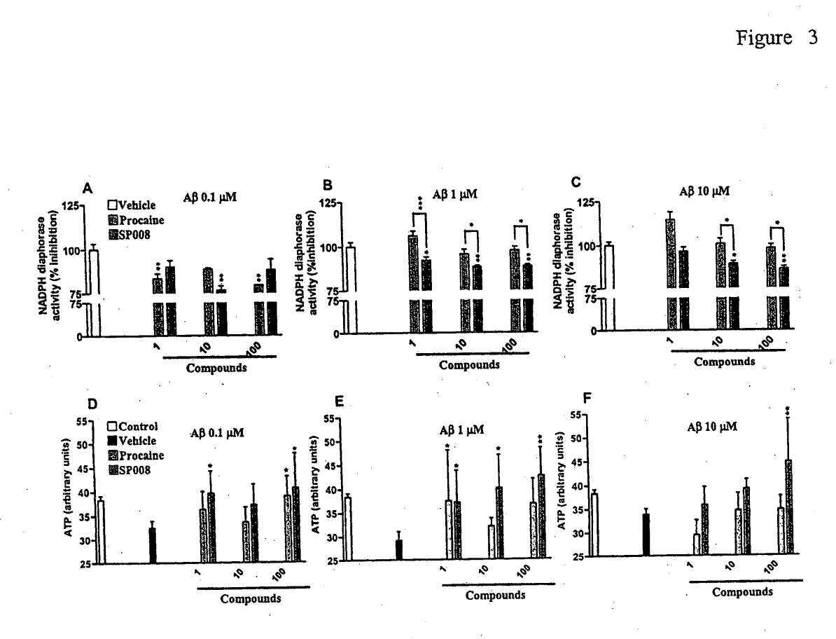

SP008 at 10 μM exerted a protective effect against 0.1 μM Aβ₁₋₄₂-induced cytotoxicity (p<0.01, n=6) (FIG. 3A) although this concentration did not preserve the Aβ₁₋₄₂-depleted ATP stock. Paradoxically, 1 and 100 μM SP008 did not reduce the 0.1 μM Aβ₁₋₄₂-induced NADPH diaphorase inhibition (FIG. 3A) but they prevented the ATP decrease (p<0.05) (FIG. 13D). SP008 demonstrated neuroprotective effects against 1 μM Aβ₁₋₄₂ assessed using the MTT assay, when used at 1 (p<0.05), 10 (p<0.01) and 100 μM (p<0.001) (FIG. 3B). This effect was accompanied by a dose-dependent ATP preservation (FIG. 3E).

SP008 administered at 10 and 100 μM concentrations displayed neuroprotective properties against 10 μM Aβ₁₋₄₂-induced toxicity in PC12 cells; this effect was statistically significant at both 10 (p<0.05, n=6) and 100 μM (p<0.01, n=6) concentrations (FIG. 3C). This effect of SP008 was accompanied by a dose-dependent restoration of ATP levels, although only the effect of 100 μM SP008 was statistically significant (p<0.01, n=6; FIG. 3F).

### Example 4

### Effect of Procaine and SP008 on Glutamate-Induced Excitotoxicity on PC12 Cells

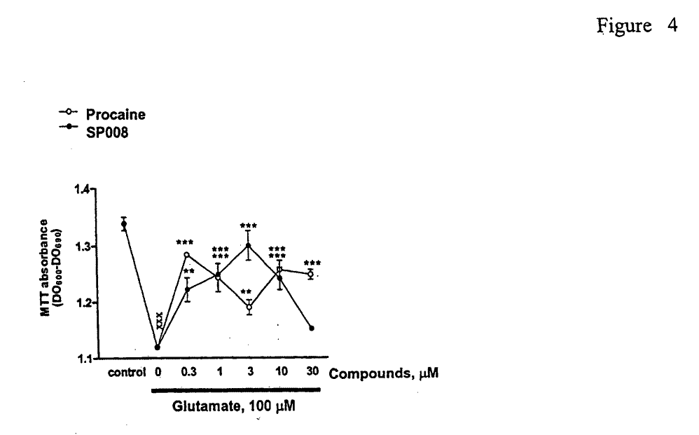

Glutamate 100 μM dramatically reduced PC12 cell viability (p<0.001, n=6; FIG. 4). Procaine prevented the glutamate-induced neurotoxicity in a biphasic manner. Two maximum effects were observed at 0.3 and 10 μM (p<0.001 compared to control, n=6). The SP008 effect was also biphasic reaching a protective peak at 3 μM (p<0.001 compared to control, n=6) followed by a decline in its neuroprotective property in the presence of at higher concentrations of glutamate. The neuroprotective effect of SP008 was more important than the procaine effect at the same concentration (p<0.001, n=6).

### DISCUSSION

During the past decades, improving the cholinergic network dysfunction associated with AD has been the main focus of the scientific community. This led to the creation of the therapeutic class of the acetylcholinesterase inhibitors (AchEI) with the tacrine as the class leader. Despite promising clinical data, the beneficial effects of tacrine were modest and the new generation of AchEI, represented by galantamine and donezepil, did not improve the delay of symptom onset compared to tacrine. This short 1-2 years delay, although priceless for the patients and their relatives, is probably due to the progressive degeneration of the cholinergic neurons and is a limitation of the use of AchEI. Even though the improvement of the cholinergic transmission of the patients suffering from AD is relevant and necessary, it is certainly not sufficient to stop or reverse the progression of the disease. Since, no major advance has been made in AD drug development, even though memantine, an antagonist of the glutamatergic NMDA-subtype receptor was recently approved to be released in the US market. The present invention provides a new class of compounds derived from the homologous domain of a series of natural compounds which were obtained by screening a database using procaine as a starting point. These molecules can protect rat pheochromocytoma PC12 cells against Aβ₁₋₄₂ neurotoxicity.

The adrenal hormone cortisol was described to worsen the AD evolution by enhancing the neuronal death, altering the mood and inducing depression and Xu et al. recently reported that a procaine-based pharmaceutical preparation reduced the stress-induced hypercorticosteronism in rat (J. Pharmacol. Exp. Ther., 307:1148 (2003)), presenting therefore procaine as an interesting approach to treat AD. However, the quick degradation of procaine into para-aminobenzoic acid and diethylaminoethanol renders it difficult to use therapeutically for AD. SP015, SP016 and SP017 were obtained by screening natural compounds database using procaine as a sub-structure (FIG. 1) and they originate from plants of the Asteraceae family, Inula britanica and Artemisia glabella. Strikingly, plants from Artemisia genus have been used traditionally as restoratives of lost or declining mental functions (Wake et al., (2000) J. Ethnopharmacol. 69:105-14).

Procaine was able to restore partially the decrease of ATP production induced by Aβ₁₋₄₂ suggesting an activity on the mitochondrial respiratory chain. Among the screened natural compounds, SP017 showed the highest protective effect on the mitochondrial function, as evidenced by the changes seen in mitochondrial diaphorase activity, with efficacy range of 30-70% of inhibition of Aβ₁₋₄₂ toxicity. Interestingly, despite the important chemical similarity between SP015 and SP016, SP016 displayed a significant effect only against low Aβ₁₋₄₂ concentrations (0.1 μM) when administered at 1 μM whereas 1 μM SP015 offered an important protection even against the highest Aβ₁₋₄₂ concentration examined. Surprisingly, the effect of these different compounds on PC12 viability after Aβ₁₋₄₂ exposure did not completely match the effect observed on the restoration of ATP content. In particular, SP015 displayed a neuroprotective effect at 1 and 10 μM only against 10 μM Aβ₁₋₄₂ while no effect was observed with SP016. This apparent discrepancy suggests that the preservation of the intracellular ATP stock is not the only mechanism by which the procaine and procaine derivatives exert their neuroprotective properties.

SP015, SP016 and SP017 chemical structures share a common 4-ethyl-1-benzoyl-piperazine substructure. The neuroprotection obtained with SP015 and SP017 and the preservation of the ATP cellular stocks induced by SP015, SP016 and SP017 against Aβ₁₋₄₂ led to the hypothesis that this common substructure might be responsible, at least in part, for the “anti-amyloid” effects disclosed herein for these natural compounds. This substructure was modified to derive the 4-ethyl-1-(2,3,4-trimethoxybenzoyl)-piperazine compound (SP008), which can be prepared in two steps.

SP008 exhibited significant neuroprotective properties against Aβ₁₋₄₂ and was more potent than procaine against the two highest concentrations of Aβ₁₋₄₂. SP008 displayed an interesting dose-effect relationship against 10 μM Aβ₁₋₄₂, predicting a lack of toxicity at high concentrations compared to SP017, the most potent natural compound of the series. The beneficial effect of SP008 on PC12 viability was further confirmed by its ability to prevent the Aβ₁₋₄₂-induced intracellular ATP stock depletion even against 10 μM Aβ₁₋₄₂. As with procaine, SP008 was able to dramatically reduce the glutamate-induced neurotoxicity on PC12 cells even when given at concentrations as low as 0.3 μM, which probably accounts for its neuroprotective effect against Aβ₁₋₄₂.

Although the possible blockade of the NMDA receptor needs to be clarified, these data suggest that SP008 shares pharmacological mechanisms with memantine, the NMDA-antagonist in use as an AD treatment. In addition, because of its common structure with procaine, SP008 might share some of the mechanisms of action of procaine.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treatment of a mammal threatened or afflicted by Alzheimer's disease, by administering to said mammal an effective amount of a compound of formula I: 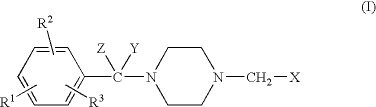
wherein:
a) R¹, R² and R³ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl, (C₁-C₆)alkylthio, thio(C₁-C₆)alkyl, (C₁-C₆)alkanoyloxy, N(R⁶)(R⁷) wherein R⁶ and R⁷ are individually H, O, (C₁-C₆) alkyl, (C₃-C₆)cycloalkyl, (C₃-C₆)cycloalkyl(C₁-C₆)alkyl, phenyl or benzyl, or R⁶ and R⁷, together with the N to which they are attached form a 5- or 6-membered ring, optionally comprising 1-2 S, N(R⁶) or nonperoxide O, or R¹ and R² together are methylenedioxy;
b) Y and Z together are ═O, —O(CH₂)ₘO— or —CH₂)ₘ— wherein m is 2-4, or Y is H and Z is OR⁹ or SR⁹, wherein R⁹ is H or (C₁-C₄)alkyl;
c) X is (C₁-C₆)alkyl, (C₁-C₆)alkoxy, hydroxyl(C₁-C₆)alkyl (C₃-C₁₂)alkenyl, (C₂-C₆)alkynyl, carboxy, (C₁-C₆)alkoxycarbonyl, thio(C₁-C₆) alkyl, (C₃-C₁₂)heterocyclo, (C₃-C₁₂) heterocycloalkyl(C₁-C₆) alkyl, aryl or heteroaryl, optionally substituted by 1, 2 or 3 R¹;
and the pharmaceutically acceptable salts thereof.

2. The method of claim 1 wherein the amount is effective to inhibit Aβ peptide-induced neurotoxicity.

3. The method of claim 1 wherein the amount is effective to inhibit Aβ₁₋₄₂ neurotoxicity.

4. The method of claim 1 wherein the amount is effective to inhibit glutamate-induced neurotoxicity in said mammal.

5. The method of claim 1 wherein the amount is effective to maintain ATP levels in neuronal cells in said mammal.

6. The method of claim 5 wherein the cells are contacted in vitro.

7. The method of claim 5 wherein the cells are contacted in vivo.

8. The method of claim 1 wherein the compound of formula I is administered to a human.

9. The method of claim 8 wherein the human is in an early stage of AD.

10. The method of claim 8 wherein the human is an AD patient.

11. The method of claim 1 wherein R¹, R² or R³ is N(R⁶)(R⁷).

12. The method of claim 1 wherein R² is (C₁-C₆)alkoxy.

13. The method of claim 1 wherein R³ is (C₁-C₆)alkoxy.

14. The method of claim 1 wherein each of R¹, R² and R³ is (C₁-C₃)alkoxy.

15. The method of claim 1 wherein Y and Z together are ═O.

16. The method of claim 1 wherein Y is H and Z is OH.

17. The method of claim 1 wherein X is (C₁-C₆)alkyl.

18. Method of claim 1 wherein X is CH₃.

19. The method of claim 1 wherein the compound of formula I is administered orally.

20. The method of claim 1 wherein the compound of formula I is administered parenterally.

21. The method of claim 1 wherein the compound of formula (I) is administered in combination with a pharmaceutically acceptable carrier.

22. The method of claim 21 wherein the carrier is a liquid, suspension or gel.

23. The method of claim 21 wherein the carrier is a solid.

24. The method of claim 1 wherein the compound of formula I is [(2,3,4-trimethoxy)phenyl]-[4-ethylpiperazin-1-yl]methanone.

25. A composition comprising a compound of formula (I) in combination with a pharmaceutically-acceptable carrier.

26. A therapeutic method to treat a neuropathy that involves a glutamate network or pathway hyperactivity comprising administering to a mammal threatened with, or afflicted by, said neuropathy, an effective amount of a compound of formula (I).

27. (canceled)


SEQUENCE LISTING (2 sequences)

SEQ ID NO: 1 - DNA, 24 bp, Artificial Sequence
  A synthetic primer
gactgtggtttgtgaagctgtcat

SEQ ID NO: 2 - DNA, 24 bp, Artificial Sequence
  A synthetic primer
aatacttctctcaccaccttggct